(19) United States
(12) Patent Application Publication
(10) Pub. No.: US 2014/0046134 A1
(43) Pub. Date: Feb. 13, 2014

(54) GUIDE SHEATH

(71) Applicant: OLYMPUS CORPORATION, Tokyo (JP)

(72) Inventor: Satoru KIKUCHI, Tokyo (JP)

(73) Assignee: OLYMPUS CORPORATION, Tokyo (JP)

(21) Appl. No.: 14/055,135

(22) Filed: Oct. 16, 2013

Related U.S. Application Data
(63) Continuation of application No. PCT/JP2012/060565, filed on Apr. 19, 2012.

(30) Foreign Application Priority Data
Apr. 25, 2011 (JP) 2011-097362

(51) Int. Cl. A61B 1/00

(52) U.S. Cl. CPC A61B 1/00154
USPC 600/114

(57) ABSTRACT

Provided is a guide sheath that is maintained in a desired shape and at a desired position even after a dilator has been pulled out therefrom so that the guide sheath can guide an insertion section to an appropriate position. A cylindrical guide sheath that guides an insertion section of a medical device to be inserted into a body cavity includes a base-end opening that is provided at a base end of a guide sheath body and into which the insertion section is inserted; a distal-end opening that is provided at a distal end of the guide sheath body and from which the insertion section extends out; and a shape maintainer that extends in an axial direction at an inner surface of the guide sheath body and has photo-curable resin that is cured by light.

### CROSS-REFERENCE TO RELATED APPLICATIONS

This is a continuation of International Application PCT/JP2012/060565, with an international filing date of Apr. 19, 2012, which is hereby incorporated by reference herein in its entirety. This application is based on Japanese Patent Application No. 2011-097362, the contents of which are incorporated herein by reference.

### TECHNICAL FIELD

The present invention relates to guide sheaths that guide insertion sections of medical devices into body cavities.

### BACKGROUND ART

In the related art, there is a known guide sheath used in the medical field for guiding an insertion section of a medical device, such as an endoscope, into a body cavity when inserting the insertion section into the body cavity (for example, see Patent Literature 1).

When such a guide sheath is to be set within a pericardial cavity, the pericardium is normally pierced before inserting a guide wire through the pericardium. Then, after inserting the guide sheath and a dilator along the guide wire, the dilator is pulled out from the guide sheath so that the guide sheath is set within the pericardial cavity.

### CITATION LIST

### Patent Literature

### PTL 1

Japanese Unexamined Patent Application, Publication No. 2003-102843

### SUMMARY OF INVENTION

### Technical Problem

Because the guide sheath disclosed in Patent Literature 1 is flexible, when the dilator is pulled out therefrom, the guide sheath may deform into an undesired shape or shift from its original position due to beating of the heart, thus making it impossible to guide the insertion section to an appropriate position.

The present invention provides a guide sheath that is maintained in a desired shape and at a desired position even after a dilator has been pulled out therefrom so that the guide sheath can guide an insertion section to an appropriate position.

### Solution to Problem

According to an aspect of the present invention, a cylindrical guide sheath that guides an insertion section of a medical device to be inserted into a body cavity includes a base-end opening that is provided at a base end of the guide sheath and into which the insertion section is inserted; a distal-end opening that is provided at a distal end of the guide sheath and from which the insertion section extends out; and a shape maintainer that extends in an axial direction at an inner surface of the guide sheath and has photo-curable resin that is cured by light.

In order to insert the insertion section of the medical device, such as an endoscope, into the body cavity, such as a pericardial cavity, by using the guide sheath according to the above aspect of the present invention, the pericardium is first pierced with a puncture needle from the skin surface, and a guide wire is then inserted into the pericardial cavity. Subsequently, the guide sheath and a dilator are inserted together into the pericardial cavity along the guide wire. Then, the dilator is pulled out so that the distal end of the guide sheath is disposed within the pericardial cavity and the base end of the guide sheath is disposed outside the body cavity. In this state, the insertion section is inserted through the base-end opening of the guide sheath so that the insertion section extending through the guide sheath extends out from the distal-end opening, whereby the insertion section is inserted into the pericardial cavity.

Subsequently, the insertion section is bent by operating the endoscope so that the guide sheath is deformed into a shape that allows for observation of an observation site. In this state, the insertion section is pulled out from the guide sheath while illumination light is radiated from the insertion section. During that time, the shape maintainer having the photo-curable resin provided at the inner surface of the guide sheath is cured due to a photo-curing reaction induced by the illumination light. As the shape maintainer provided at the inner surface of the guide sheath becomes cured, the guide sheath becomes fixed in a desired shape. Accordingly, the guide sheath can be maintained in a shape that allows for observation of the observation site, so that the guide sheath can be prevented from deforming into an undesired shape or from shifting in an undesired direction due to, for example, beating of the heart, whereby the guide sheath can properly be brought near the observation or treatment site. Consequently, the observation site can be properly observed and treated.

In the above aspect, the shape maintainer may be formed of a porous material containing the photo-curable resin.

With this configuration, the photo-curable resin can be retained by the porous material so that the photo-curable resin can be uniformly distributed at the inner surface of the guide sheath. Consequently, the guide sheath can be effectively fixed so that the guide sheath can be maintained in a shape that allows for observation of the observation site.

In the above aspect, the shape maintainer may be formed of a hollow permeable member through which the photo-curable resin flows.

With this configuration, the photo-curable resin is injected into the hollow permeable member from the outside so that the hollow permeable member can be filled with the photo-curable resin. Thus, the photo-curable resin can be injected into the guide sheath after the guide sheath is given a desired shape, thereby preventing the photo-curable resin from being cured due to undesired light (such as external light entering the guide sheath when the guide sheath is being stored or external light entering the guide sheath when the guide sheath is being inserted into the body cavity). In other words, this can facilitate the storing process of the guide sheath or the insertion process of the guide sheath into the body cavity.

In the above aspect, the shape maintainer may be partially disposed in the axial direction of the guide sheath.

With this configuration, the guide sheath can be cured partially in the axial direction in accordance with, for example, the shape of the body cavity. This improves the ease of use when inserting the endoscope insertion section into the guide sheath. Furthermore, for example, by making the sections in contact with the pericardium and the heart free of the shape maintainer (that is, by making these sections non-curable), the load on the human body can be reduced.

In the above aspect, the photo-curable resin may be cured by light with a wavelength ranging between 380 nm and 700 nm.

Accordingly, the photo-curable resin can be cured with visible light (i.e., light with a wavelength ranging between 380 nm and 700 nm) generally used as illumination light in an endoscope. This eliminates the need to provide an additional light source for curing the photo-curable resin.

In the above aspect, the guide sheath may further include a light guide section that is provided at the inner surface of the guide sheath and guides light, which enters the guide sheath from the base end thereof, in the axial direction of the guide sheath; and a light distributing section that is provided at the inner surface of the guide sheath and distributes the light guided by the light guide section over an entire surface of the shape maintainer.

With this configuration, when light enters the light guide section, provided at the inner surface of the guide sheath, from the base end of the guide sheath, the light is guided in the axial direction of the guide sheath by the light guide section. The light guided in the axial direction of the guide sheath is uniformly distributed over the entire surface of the shape maintainer by the light distributing section. Accordingly, the shape maintainer having the photo-curable resin can be cured without having to pull out the endoscope insertion section from the guide sheath, so that the guide sheath can be fixed in a desired shape. Consequently, the guide sheath can be prevented from deforming or from being positionally displaced when pulling out the endoscope insertion section therefrom, whereby the guide sheath can be fixed at a more appropriate position.

In the above aspect, the guide sheath may further include a light source that is provided at the base end of the guide sheath and emits illumination light toward the light guide section.

With this configuration, the illumination light from the light source can be guided in the axial direction of the guide sheath by the light guide section so that the shape maintainer having the photo-curable resin can be cured efficiently, whereby the guide sheath can be fixed in a desired shape.

In the above aspect, the light guide section and the light distributing section may be provided around the entire circumference of the inner surface of the guide sheath, and the light source may include a plurality of light sources that are spaced apart from each other in a circumferential direction of the inner surface of the guide sheath.

With this configuration, illumination light is emitted from the plurality of light sources spaced apart from each other in the circumferential direction of the inner surface of the guide sheath, and the illumination light can be guided to the shape maintainer by the light guide section and the light distributing section provided around the entire circumference of the inner surface of the guide sheath. Consequently, the shape maintainer having the photo-curable resin can be cured efficiently, whereby the guide sheath can be fixed in a desired shape.

In the above aspect, the guide sheath may further include a sheet-like detachable section having an adhesive layer and provided between the inner surface of the guide sheath and the shape maintainer; and a detachment manipulation section that is connected to the detachable section at the distal end of the guide sheath and extends through the guide sheath to the base end of the guide sheath.

With this configuration, when the shape maintainer is cured and the guide sheath is to be pulled out from the pericardial cavity, the shape maintainer can be detached by pulling on the detachment manipulation section extending to the base end of the guide sheath. Consequently, the shape maintainer provided over the surface of the detachable section can be simultaneously detached from the inner surface of the guide sheath. Accordingly, the guide sheath can be given flexibility again so that the load on the human body (such as the pericardium) when pulling out the guide sheath from the pericardial cavity can be reduced, thereby allowing for improved ease of use and improved safety when pulling out the guide sheath.

### Advantageous Effects of Invention

The present invention is advantageous in that the guide sheath can be maintained in a desired shape and at a desired position even after the dilator has been pulled out therefrom so that the guide sheath can guide the insertion section to an appropriate position.

### BRIEF DESCRIPTION OF DRAWINGS

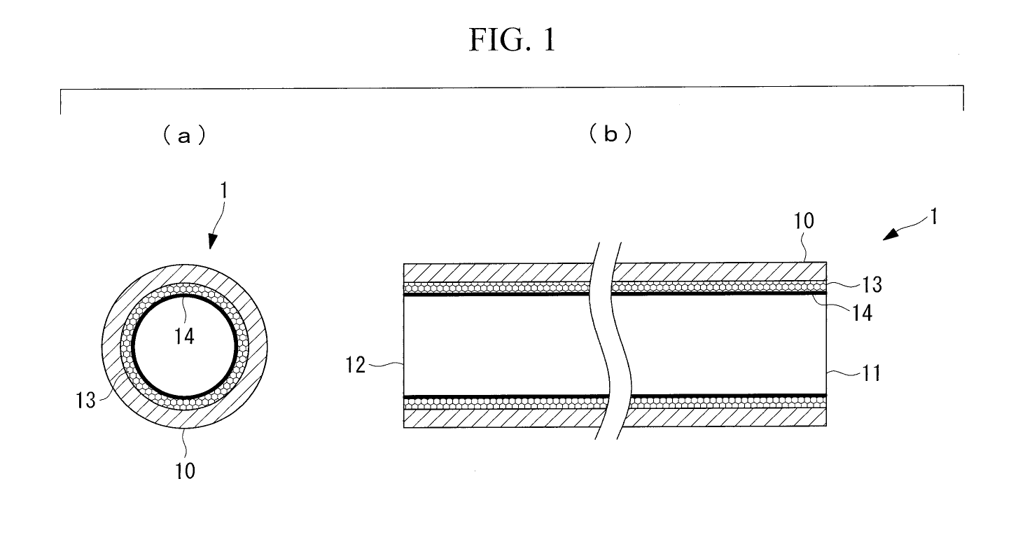

{FIG. 1} FIG. 1 schematically illustrates the configuration of a guide sheath according to a first embodiment of the present invention and includes a cross-sectional view (a) and a vertical sectional view (b).

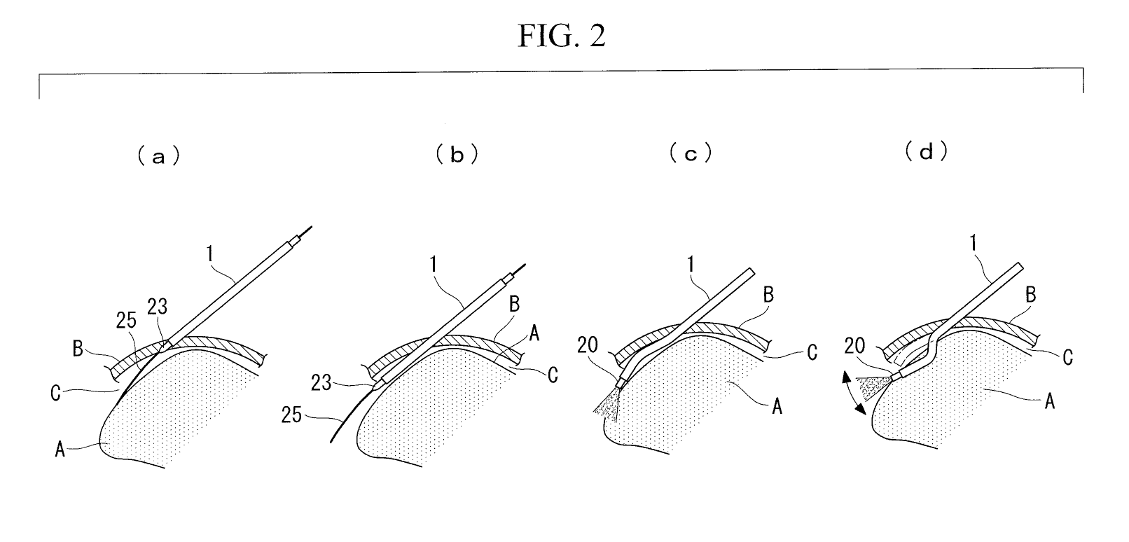

{FIG. 2} FIG. 2 illustrates a state where the guide sheath in FIG. 1 is being inserted into a pericardial cavity.

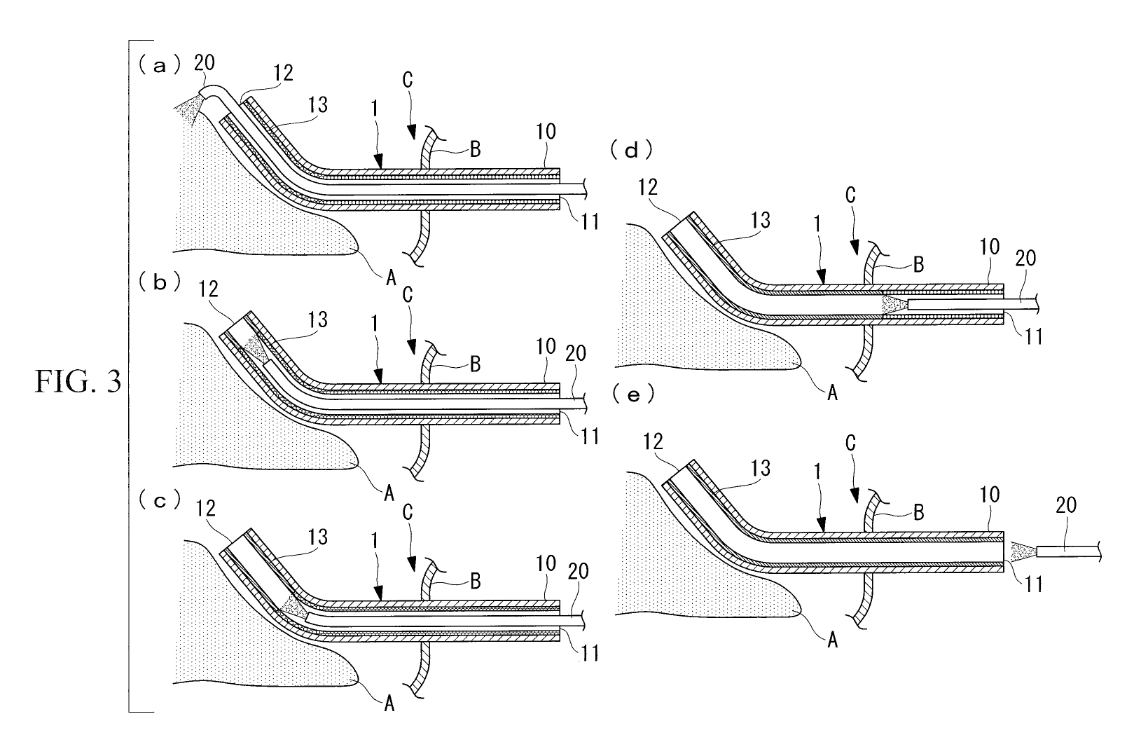

{FIG. 3} FIG. 3 illustrates a state where the guide sheath in FIG. 1 is being fixed within the pericardial cavity.

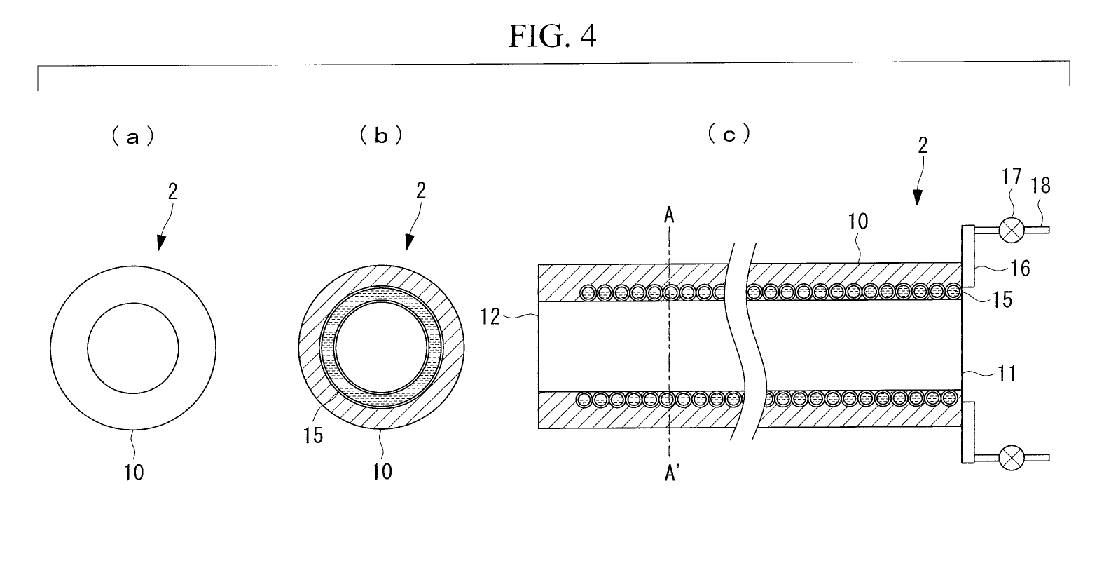

{FIG. 4} FIG. 4 schematically illustrates a guide sheath according to a second embodiment of the present invention and includes a front view (a) as viewed from the distal end, a cross-sectional view (b) taken along line A-A′, and a vertical sectional view (c).

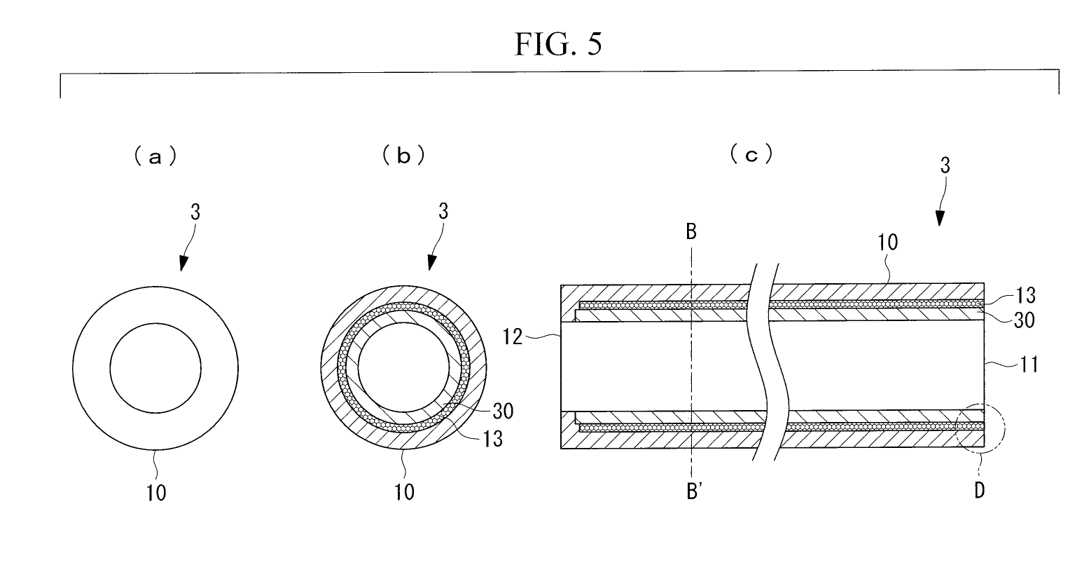

{FIG. 5} FIG. 5 schematically illustrates a guide sheath according to a third embodiment of the present invention and includes a front view (a) as viewed from the distal end, a cross-sectional view (b) taken along line B-B′, and a vertical sectional view (c).

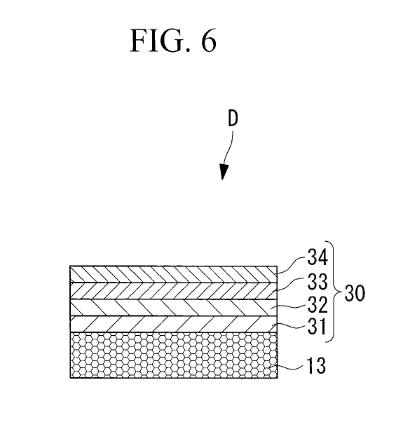

{FIG. 6} FIG. 6 is a partial enlarged view of area D of the guide sheath shown in FIG. 5.

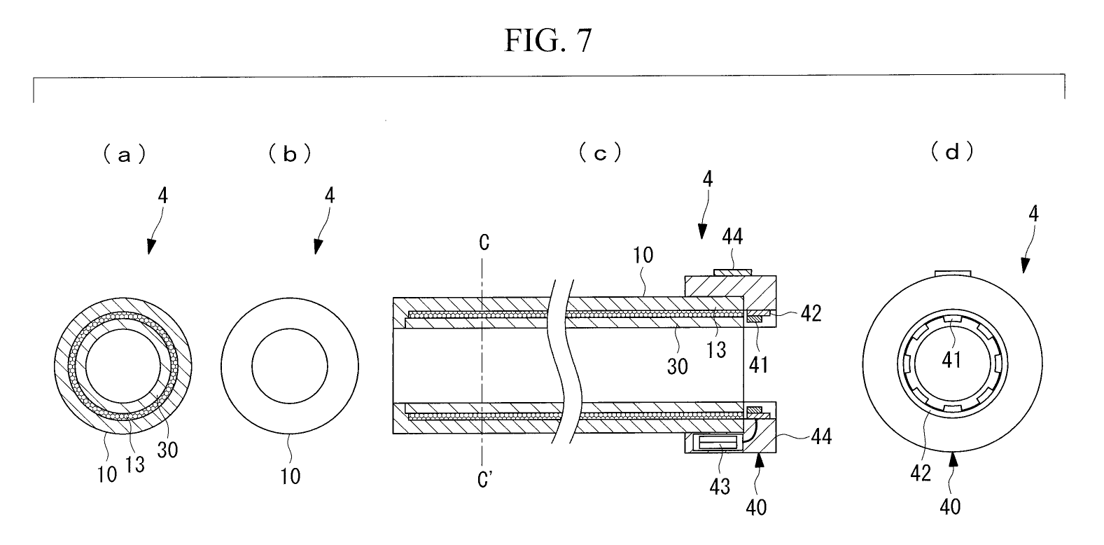

{FIG. 7} FIG. 7 schematically illustrates a guide sheath according to a modification of FIG. 5 and includes a cross-sectional view (a) taken along line C-C′, a front view (b) as viewed from the distal end, a vertical sectional view (c), and a front view (d) as viewed from the base end.

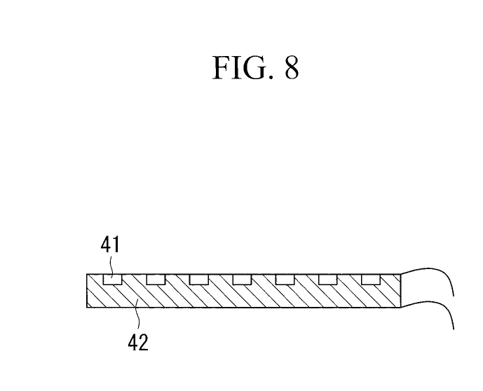

{FIG. 8} FIG. 8 is a partial enlarged view of a substrate in the guide sheath in FIG. 7.

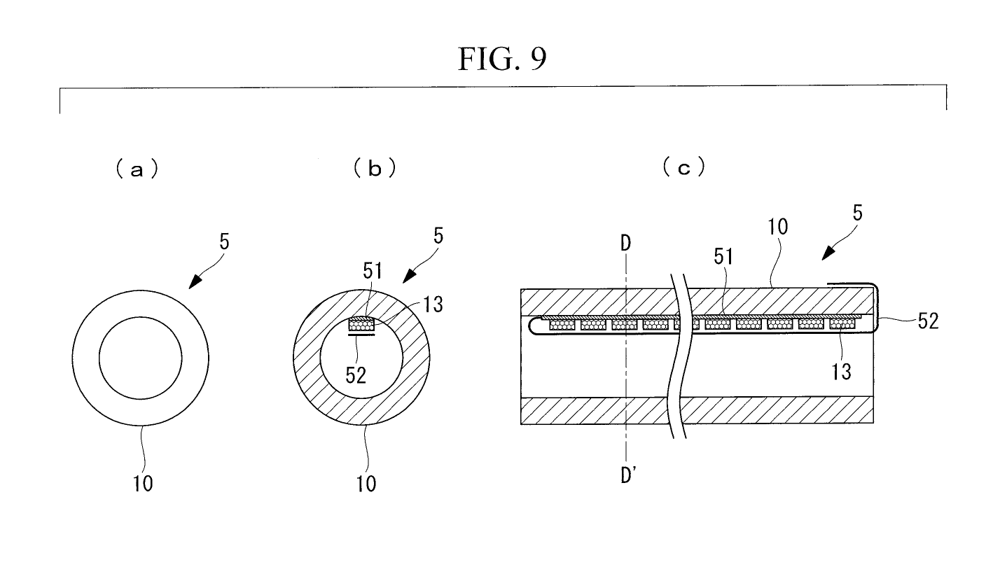

{FIG. 9} FIG. 9 schematically illustrates a guide sheath according to a fourth embodiment of the present invention and includes a front view (a) as viewed from the distal end, a cross-sectional view (b) taken along line D-D′, and a vertical sectional view (c).

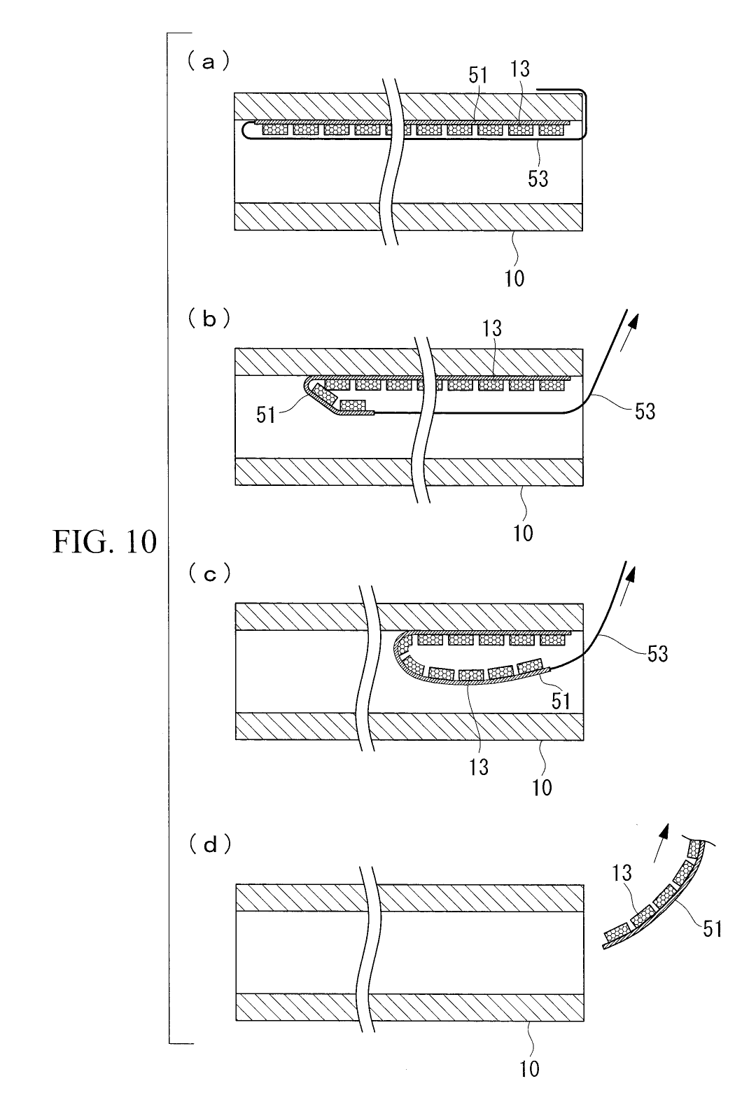

{FIG. 10} FIG. 10 illustrates a state where a shape maintainer is being detached from a guide sheath body in the guide sheath in FIG. 9.

### DESCRIPTION OF EMBODIMENTS

### First Embodiment

A guide sheath 1 according to a first embodiment of the present invention will be described below with reference to the drawings.

As shown in FIGS. 1(a) and 1(b), the guide sheath 1 according to this embodiment is a cylindrical guide sheath that guides, for example, an insertion section of a medical device to be inserted into a body cavity. The following description relates to an example where an endoscope insertion section 20 (see FIG. 3) is inserted into the pericardial cavity C formed between the heart A and the pericardium B by using the guide sheath 1 according to this embodiment, as shown in FIGS. 2(a) to 2(d). When inserting the guide sheath 1 into the pericardial cavity C, which will be described later, a dilator 23 and a guide wire 25 inserted into the guide sheath 1 are used.

As shown in FIGS. 1(a) and 1(b), the guide sheath 1 according to this embodiment includes a cylindrical guide sheath body 10, a shape maintainer 13 provided at the inner surface of the guide sheath body 10, and a protection cover 14 provided over the surface of the shape maintainer 13.

As shown in FIG. 1(b), the guide sheath body 10 is a flexible tube-like cylindrical member composed of, for example, plastic. The guide sheath body 10 has a base-end opening 11 provided at the base end of the guide sheath body 10 and a distal-end opening 12 provided at the distal end of the guide sheath body 10.

The base-end opening 11 is an opening provided at the base end of the guide sheath body 10. As shown in FIGS. 2(a) to 2(d), the endoscope insertion section 20, the dilator 23, and the guide wire 25 are inserted through the base-end opening 11.

The distal-end opening 12 is an opening provided at the distal end of the guide sheath body 10. As shown in FIGS. 2(a) to 2(d), the endoscope insertion section 20, the dilator 23, and the guide wire 25 extending through the guide sheath body 10 extend out from the distal-end opening 12.

The shape maintainer 13 is provided continuously in the axial direction and around the entire circumference of the inner surface of the guide sheath body 10, and contains photo-curable resin that is curable with light. Specifically, the shape maintainer 13 is formed of a porous material (such as a sponge-like member) having a certain thickness in the radial direction of the guide sheath body 10. With this configuration, the photo-curable resin can be uniformly distributed within the shape maintainer 13.

Examples of the photo-curable resin include epoxy-based photo-curable resin and acrylic-based photo-curable resin, and transparent and colorless resin is preferred.

Furthermore, the photo-curable resin is curable with visible light (such as light with a wavelength ranging between 380 nm and 700 nm), and is cured with illumination light radiated from the tip of the endoscope insertion section 20.

The protection cover 14 is a transparent film that protects the shape maintainer 13 from the endoscope insertion section 20, the dilator 23, and the guide wire 25 when these components are inserted into the guide sheath 1.

The dilator 23 is a rod-shaped member that is inserted into the guide sheath 1 through the base-end opening 11. The dilator 23 is inserted into the guide sheath 1 along the guide wire 25 set inside the guide sheath 1 in advance. The dilator 23 has a tapered end such that the dilator 23 is capable of penetrating the pericardium while expanding a hole therein. In order to minimize invasiveness to biological tissue within the body cavity, the dilator 23 is desirably composed of biocompatible resin.

The guide wire 25 is inserted into the guide sheath 1 through the base-end opening 11, extends out from the distal-end opening 12, and is inserted into the pericardial cavity C. The guide wire 25 guides the dilator 23 from the base-end opening 11 to the distal-end opening 12 of the guide sheath 1. Moreover, the guide wire 25 also guides the dilator 23 and the guide sheath 1 to the pericardial cavity C.

The following description relates to the operation performed when inserting the endoscope insertion section 20 into the pericardial cavity by using the guide sheath 1 having the above-described configuration.

In order to insert the endoscope insertion section 20 into the pericardial cavity C shown in FIGS. 2(a) to 2(d), the pericardium B is first pierced with a puncture needle from the skin surface, and the guide wire 25 is then inserted into the pericardial cavity C through the puncture needle.

Subsequently, the guide wire 25 is inserted further into the pericardial cavity C so that the tip of the guide wire 25 is disposed near an observation site in the pericardial cavity C. Then, the puncture needle is pulled out from the pericardium B.

Subsequently, as shown in FIG. 2(a), the guide sheath 1 and the dilator 23 are inserted together into the pericardial cavity C along the guide wire 25. In this case, the guide sheath 1, having flexibility, is set in a linear shape along the dilator 23 due to the rod-shaped dilator 23 extending from the base-end opening 11 to the distal-end opening 12.

Thus, the guide sheath 1 and the dilator 23 can be readily inserted into the pericardial cavity C along the guide wire 25. Accordingly, as shown in FIG. 2(b), the distal ends of the guide sheath 1 and the dilator 23 are disposed near the observation site in the pericardial cavity C.

Subsequently, the dilator 23 is pulled out from the guide sheath 1 so that the distal end of the guide sheath 1 is disposed near the observation site within the pericardial cavity C and the base end of the guide sheath 1 is disposed outside the body cavity. Thus, the endoscope insertion section 20 can be guided into the pericardial cavity from outside the body cavity.

In this state, the endoscope insertion section 20 is inserted through the base-end opening 11 of the guide sheath 1, as shown in FIG. 2(c), so that the endoscope insertion section 20 extending through the guide sheath 1 extends out from the distal-end opening 12, whereby the endoscope insertion section 20 is inserted into the pericardial cavity

C.

Subsequently, the insertion section 20 is bent by a bending mechanism included in the endoscope so that the guide sheath body 10 is deformed into a shape that allows for observation of the observation site. Thus, the endoscope insertion section 20 is guided to a position near the observation site in the pericardial cavity C so that the observation site can be observed and treated.

However, because the guide sheath 1 is flexible in this state, as shown in FIG. 2(d), the guide sheath 1 may deform into an undesired shape or shift from its original position due to beating of the heart A, possibly resulting in deviation from the observation site.

With regard to the guide sheath 1 according to this embodiment, in the state where the guide sheath 1 and the endoscope insertion section 20 are inserted in the pericardial cavity C, as shown in FIG. 3(a), the insertion section 20 is pulled out from the guide sheath 1 while illumination light is radiated from the insertion section 20, as shown in FIGS. 3(b) to 3(e). During that time, the shape maintainer 13 having the photo-curable resin and provided at the inner surface of the guide sheath body 10 is cured due to a photo-curing reaction induced by the illumination light from the insertion section 20.

As the shape maintainer 13 provided at the inner surface of the guide sheath body 10 becomes cured, the guide sheath 1 becomes fixed into a desired shape. Accordingly, the guide sheath 1 can be maintained in a shape that allows for observation of the observation site, so that the guide sheath 1 can be prevented from deforming into an undesired shape or from shifting in an undesired direction due to beating of the heart A, whereby the endoscope insertion section 20 can be guided to an appropriate position. Consequently, the observation site can be properly observed and treated.

Furthermore, with the guide sheath 1 according to this embodiment, since the shape maintainer 13 is formed of a porous material containing photo-curable resin, the photo-curable resin can be retained by the porous material so that even when the guide sheath 1 is set or deformed at any angle, the photo-curable resin can be uniformly distributed at the inner surface of the guide sheath body 10. Consequently, the guide sheath 1 can be effectively fixed so that the guide sheath 1 can be maintained in a shape that allows for observation of the observation site.

Furthermore, since the photo-curable resin used is curable with visible light (such as light with a wavelength ranging between 380 nm and 700 nm), the photo-curable resin can be cured with illumination light from a commonly-used endoscope. This eliminates the need to provide an additional light source for curing the photo-curable resin.

In this embodiment, the shape maintainer 13 is described as being provided continuously in the axial direction and around the entire circumference of the inner surface of the guide sheath body 10. Alternatively, the shape maintainer 13 may be disposed partially in the axial direction or the circumferential direction of the guide sheath body 10.

With this configuration, the guide sheath 1 can be cured partially in the axial direction in accordance with, for example, the shape of the body cavity. This improves the ease of use when inserting the endoscope insertion section 20 into the guide sheath 1. Furthermore, for example, by making the sections in contact with the pericardium B and the heart A free of the shape maintainer 13 (that is, by making these sections non-curable), the load on the human body can be reduced.

### Second Embodiment

A guide sheath 2 according to a second embodiment will be described below with reference to the drawings. In the following description, components of the guide sheath according to this embodiment that are the same as those in the guide sheath 1 according to the first embodiment are given the same reference characters, and descriptions thereof will be omitted. The following description will mainly be directed to differences from the guide sheath 1 according to the first embodiment.

As shown in FIGS. 4(a) to 4(c), the guide sheath 2 according to this embodiment is a cylindrical guide sheath that guides, for example, an insertion section of a medical device to be inserted into a body cavity, and includes a cylindrical guide sheath body 10, a shape maintainer 15 provided at the inner surface of the guide sheath body 10, a pipe 16 and a valve 17 that are connected to the shape maintainer 15, and a photo-curable-resin injection port 18.

The shape maintainer 15 is a hollow pipe constituted of a flexible permeable member composed of, for example, transparent plastic and is formed into a helical shape at the inner surface of the guide sheath body 10.

The shape maintainer 15 is connected to the photo-curable-resin injection port 18 via the pipe 16 and the valve 17 provided outside (i.e., at the base end of) the guide sheath body 10. With this configuration, by opening the valve 17 and then injecting photo-curable resin through the photo-curable-resin injection port 18 by using, for example, an injector, the shape maintainer 15 can be filled with the photo-curable resin.

With the guide sheath 2 according to this embodiment having the above-described configuration, the photo-curable resin is injected into the hollow permeable member (i.e., the shape maintainer 15) from the outside so that the hollow permeable member can be filled with the photo-curable resin. Thus, the photo-curable resin can be injected into the guide sheath 2 after the guide sheath 2 is given a desired shape, thereby preventing the photo-curable resin from being cured due to undesired light (such as external light entering the guide sheath 2 when it is being stored or external light entering the guide sheath 2 when it is being inserted into the body cavity). In other words, this can facilitate the storing process of the guide sheath 2 or the insertion process of the guide sheath 2 into the body cavity.

Although the shape maintainer 15 is described as being a helical pipe in this embodiment, the shape maintainer 15 may have any shape so long as it can be filled with the photo-curable resin. Therefore, as an alternative to the above-described shape, the shape maintainer 15 may be a rectangular or elliptical pipe extending in the axial direction of the guide sheath 2 or may be a bag that expands by being filled with the photo-curable resin.

### Third Embodiment

A guide sheath 3 according to a third embodiment will be described below with reference to the drawings. In the following description, components of the guide sheath 3 according to this embodiment that are the same as those in the guide sheath according to each of the above embodiments are given the same reference characters, and descriptions thereof will be omitted. The following description will mainly be directed to differences from the guide sheath according to each of the above embodiments.

As shown in FIGS. 5(a) to 5(c), the guide sheath 3 according to this embodiment is a cylindrical guide sheath that guides, for example, an insertion section of a medical device to be inserted into a body cavity, and includes a cylindrical guide sheath body 10, a shape maintainer 13 provided at the inner surface of the guide sheath body 10, and a light guide unit 30 provided over the surface of the shape maintainer 13.

FIG. 6 is a partial enlarged view of area D in FIG. 5(c).

As shown in FIG. 6, the light guide unit 30 is formed by laminating a diffusing film 31, a light-distribution control sheet (i.e., a light distributing section) 32, a light guide layer (i.e., a light guide section) 33, and a mirror film 34 in this order from the surface of the shape maintainer 13.

The light guide layer 33 is composed of, for example, transparent plastic and guides light entering the base end of the guide sheath 3 toward the distal end of the guide sheath 3 in the axial direction while internally reflecting the light.

The mirror film 34 reflects the light from the light guide layer 33 and makes the light enter the light guide layer 33 again.

The light-distribution control sheet 32 distributes the light guided by the light guide layer 33 uniformly over the entire surface of the shape maintainer 13.

The diffusing film 31 diffuses the light transmitted through the light-distribution control sheet 32 and radiates the light toward the shape maintainer 13.

The operation of the guide sheath 3 according to this embodiment having the above-described configuration will be described below.

First, illumination light is radiated onto an entrance surface (i.e., the base end) of the light guide unit 30 provided at the base end of the guide sheath 3. The illumination light radiated onto the entrance surface of the light guide unit 30 is guided toward the distal end of the guide sheath 3 in the axial direction while repeatedly undergoing internal reflection within the light guide layer 33 provided between the mirror film 34 and the light-distribution control sheet 32.

Of the light guided through the light guide layer 33, the light traveling toward the mirror film 34 (i.e., inward in the radial direction) is reflected toward the shape maintainer 13 (i.e., outward in the radial direction) by the mirror film 34. On the other hand, the light traveling toward the shape maintainer 13 (i.e., outward in the radial direction) is actively guided to the shape maintainer 13 by the light-distribution control sheet 32.

Accordingly, with the guide sheath 3 according to this embodiment, the light entering the light guide unit 30 is guided toward the distal end of the guide sheath 3 in the axial direction by the light guide layer 33, while a portion of the light guided by the light guide layer 33 is radiated in a direction (i.e., outward in the radial direction) orthogonal to the axis of the guide sheath 3, so that the illumination light can be guided uniformly over the entire surface of the shape maintainer 13.

Accordingly, the shape maintainer 13 having the photo-curable resin can be cured without having to pull out the endoscope insertion section 20 from the guide sheath 3, so that the guide sheath 3 can be fixed in a desired shape. Consequently, the guide sheath 3 can be prevented from deforming or from being positionally displaced when pulling out the endoscope insertion section 20 therefrom, whereby the guide sheath 3 can be fixed at a more appropriate position.

### Modification

As a modification of the guide sheath 3 according to this embodiment, a light source unit 40 may be provided at the base end of the guide sheath, as shown in FIGS. 7(a) to 7(d). In the following description, components of a guide sheath 4 according to this modification that are the same as those in the guide sheath 3 according to the above embodiment are given the same reference characters, and descriptions thereof will be omitted. The following description will mainly be directed to differences from the guide sheath 3 according to the above embodiment.

As shown in FIGS. 7(a) to 7(d), the guide sheath 4 according to this modification is a cylindrical guide sheath that guides, for example, an insertion section of a medical device to be inserted into a body cavity, and includes a cylindrical guide sheath body 10, a shape maintainer 13 provided at the inner surface of the guide sheath body 10, a light guide unit 30 provided over the surface of the shape maintainer 13, and a light source unit 40 provided at the base end of the guide sheath.

The light guide unit 30 is provided around the entire circumference of the inner surface of the guide sheath body 10.

The light source unit 40 includes a plurality of LEDs (light sources) 41, a substrate 42, a battery 43, a switch 44, and a light-source-unit body 44.

The light-source-unit body 44 is a cylindrical member connected to the base end of the guide sheath.

Referring to FIG. 8, the substrate 42 is composed of a bendable flexible material and has the plurality of LEDs 41 arranged thereon. The substrate 42 is joined to the inner peripheral surface of the light-source-unit body 44.

With this configuration, the plurality of LEDs 41 are spaced apart from each other in the circumferential direction of the inner surface of the guide sheath body 10, as shown in FIG. 7(d). Furthermore, the plurality of LEDs 41 are disposed such that the optical axes thereof are oriented toward the light guide unit 30.

The battery 43 is connected to the plurality of LEDs 41 and supplies electric power to the LEDs 41.

The switch 44 is used for controlling the on/off state of the supply of electric power from the battery 43 to the LEDs 41.

With the guide sheath 4 according to this modification having the above-described configuration, illumination light is emitted from the plurality of LEDs 41 spaced apart from each other in the circumferential direction of the inner surface of the guide sheath body 10, and the illumination light can be made to enter the light guide unit 30. Because the light guide unit 30 is provided around the entire circumference of the inner surface of the guide sheath body 10, the illumination light from the light source unit 40 can be guided uniformly to the entire shape maintainer 13 by the light guide unit 30 (i.e., the light guide layer 33 and the light-distribution control sheet 32). Consequently, the shape maintainer 13 having the photo-curable resin can be cured efficiently so that the guide sheath 4 can be fixed in a desired shape.

### Fourth Embodiment

A guide sheath 5 according to a fourth embodiment will be described below with reference to the drawings. In the following description, components of the guide sheath 5 according to this embodiment that are the same as those in the guide sheath according to each of the above embodiments are given the same reference characters, and descriptions thereof will be omitted. The following description will mainly be directed to differences from the guide sheath according to each of the above embodiments.

As shown in FIGS. 9(a) to 9(c), the guide sheath 5 according to this embodiment includes a detachable section 51 provided between the inner surface of the guide sheath body 10 and the shape maintainer 13 and a detachment manipulation section (detachment manipulation section) 52 connected to the detachable section 51 at the distal end of the guide sheath body 10.

The detachable section 51 is a sheet-like detachable section having adhesive layers. One surface of the detachable section 51 is joined to the inner surface of the guide sheath body 10 by means of the adhesive force of the corresponding adhesive layer. The other surface of the detachable section 51 is joined to the shape maintainer 13 by means of the adhesive force of the corresponding adhesive layer.

The detachment manipulation section 52, which is in the form of a string, is connected to the detachable section 51 at the distal end of the guide sheath body 10 and extends through the guide sheath body 10 to the base end of the guide sheath body 10.

In this embodiment, in order to facilitate the detachment of the detachable section 51 and the shape maintainer 13 joined to the surface thereof, the shape maintainer 13 is partially provided at the inner surface of the guide sheath body 10 in the circumferential direction and the axial direction thereof. Alternatively, the shape maintainer 13 may extend continuously in the axial direction of the guide sheath body 10. As another alternative, a plurality of shape maintainers 13 may be spaced apart from each other in the circumferential direction of the guide sheath body 10.

The following description relates to the operation of the guide sheath 5 according to this modification having the above-described configuration when pulling out the guide sheath 5 from the pericardial cavity C.

Referring to FIG. 10(a), with the guide sheath 5 according to this modification, the endoscope insertion section 20 is pulled out from the guide sheath 5 while illumination light is emitted from the endoscope insertion section 20, so that the shape maintainer 13 having the photo-curable resin becomes cured.

Referring to FIGS. 10(b) to 10(d), in this state, the shape maintainer 13 can be detached by pulling, toward the base end, the detachment manipulation section 52 extending to the base end of the guide sheath body 10. Specifically, when the tensile force produced by pulling the detachment manipulation section 52 becomes greater than the adhesive force applied by the adhesive layer between the guide sheath body 10 and the detachable section 51, the adhesive layer of the detachable section 51 becomes detached from the inner surface of the guide sheath body 10. At the same time, the shape maintainer 13 joined to the surface of the detachable section 51 also becomes detached from the inner surface of the guide sheath body 10.

Accordingly, the guide sheath 5 can be given flexibility again so that the load on the human body (such as the pericardium B) when pulling out the guide sheath 5 from the pericardial cavity C can be reduced, thereby allowing for improved ease of use and improved safety when pulling out the guide sheath 5.

Although the embodiments and the modification according to the present invention have been described in detail above with reference to the drawings, the specific configurations are not limited to these embodiments. For example, design modifications are included in the scope of the invention so long as they do not depart from the scope of the invention. For example, the present invention may be applied to embodiments achieved by appropriately combining these embodiments and modifications.

Furthermore, although the description of each of the above embodiments is directed to an example where an endoscope insertion section is inserted into a pericardial cavity by using the guide sheath according to the present invention, the present invention is not limited to this example. The guide sheath according to the present invention may be inserted into a different body cavity.

### REFERENCE SIGNS LIST

A heart
B pericardium
C pericardial cavity
1, 2, 3, 4, 5 guide sheath
10 guide sheath body
11 base-end opening
12 distal-end opening
13 shape maintainer
14 protection cover
15 shape maintainer
20 insertion section
23 dilator
25 guide wire
30 light guide unit
31 diffusing film
32 light-distribution control sheet (light distributing section)
33 light guide layer (light guide section)
34 mirror film
40 light source unit
41 LED (light source)
42 substrate
51 detachable section
52 detachment manipulation section

1. A cylindrical guide sheath that guides an insertion section of a medical device to be inserted into a body cavity, the guide sheath comprising:
a base-end opening that is provided at a base end of the guide sheath and into which the insertion section is inserted;
a distal-end opening that is provided at a distal end of the guide sheath and from which the insertion section extends out; and
a shape maintainer that extends in an axial direction at an inner surface of the guide sheath and has photo-curable resin that is cured by light.

2. The guide sheath according to claim 1,
wherein the shape maintainer is formed of a porous material containing the photo-curable resin.

3. The guide sheath according to claim 1,
wherein the shape maintainer is formed of a hollow permeable member through which the photo-curable resin flows.

4. the guide sheath according to claim 1,
wherein the shape maintainer is partially disposed in the axial direction of the guide sheath.

5. The guide sheath according to claim 1, wherein the photo-curable resin is cured by light with a wavelength ranging between 380 nm and 700 nm.

6. The guide sheath according to claim 1, further comprising:
a light guide section that is provided at the inner surface of the guide sheath and guides light, which enters the guide sheath from the base end thereof, in the axial direction of the guide sheath; and
a light distributing section that is provided at the inner surface of the guide sheath and distributes the light guided by the light guide section over an entire surface of the shape maintainer.

7. The guide sheath according to claim 6, further comprising:
a light source that is provided at the base end of the guide sheath and emits illumination light toward the light guide section.

8. the guide sheath according to claim 7,
wherein the light guide section and the light distributing section are provided around an entire circumference of the inner surface of the guide sheath, and
wherein the light source includes a plurality of light sources that are spaced apart from each other in a circumferential direction of the inner surface of the guide sheath.

9. The guide sheath according to claim 1, further comprising:
a sheet-like detachable section having an adhesive layer and provided between the inner surface of the guide sheath and the shape maintainer; and
a detachment manipulation section that is connected to the detachable section at the distal end of the guide sheath and extends through the guide sheath to the base end of the guide sheath.
